(19) United States
(12) Patent Application Publication
(10) Pub. No.: US 2025/0186772 A1
(43) Pub. Date: Jun. 12, 2025

(54) DEVICES AND METHODS FOR TREATMENT OF ANXIETY- AND STRESS-RELATED DISORDERS USING VESTIBULAR NERVE STIMULATION

(71) Applicant: Neurovalens Limited, Portglenone (GB)

(72) Inventor: Jason McKEOWN, Portglenone (GB)

(21) Appl. No.: 19/060,379

(22) Filed: Feb. 21, 2025

Related U.S. Application Data
(63) Continuation-in-part of application No. 17/207,491, filed on Mar. 19, 2021.
(60) Provisional application No. 62/992,020, filed on Mar. 19, 2020.

(51) Int. Cl. A61M 21/02

(52) U.S. Cl. CPC A61M 21/02

(57) ABSTRACT

Electrical stimulation of the vestibular nerve system is applied via electrodes attached to a current source to influence the neurological components of the autonomic nervous system and reduce symptoms of an anxiety- and/or stress-related disorders in a subject. Treatment with a VeNS device may be applied in response to a triggering event or may be performed regularly for a fixed period of time over an extended treatment period until a desired level of improvement in disorder symptoms has been achieved.

### RELATED APPLICATIONS

This is a continuation-in part of application Ser. No. 17/207,491, filed Mar. 19, 2021, which claims the benefit of the priority of Provisional Application No. 62/992,020, filed Mar. 19, 2020, each of which is incorporated herein by reference in its entirety.

### FIELD OF THE INVENTION

Systems, methods and devices provided herein relate to vestibular stimulation, and more specifically to stimulating the vestibular nucleus to treat anxiety- and stress-related disorders.

### BACKGROUND

The brainstem controls many involuntary autonomic functions of the body including blood pressure, heart rate, breathing, balance, and sleep. Additionally, the brain regulates stress and behavior through physiological reactions that are often complex and not yet fully understood. Anxiety is one of these stress-related mental and physiological responses that begins in the brain and cascades throughout the body into various physiological changes that may include increased heart rate, rapid breathing, sweating, nausea, weakness, fainting, and more. Key areas of the brain thought to influence anxiety include the hypothalamus, the suprachiasmatic nucleus (SCN) and the locus coeruleus (LC).

Although anxiety can be a normal, healthy response to certain types of external stress, chronic or uncontrolled anxiety can form the basis for mental and physiological disorders which may lead to continuing mental and physical health issues. Conditions arising from an extremely stressful or terrifying event, known as “post-traumatic stress disorder”, or “PTSD”, can be debilitating, with symptoms including flashbacks, nightmares, severe anxiety, and uncontrollable thoughts about the event. PTSD is not universally considered an anxiety-related disorder—in some regions is has been associated with trauma and stressor-related disorders. Nonetheless, similarities exist in consideration of diagnosis, treatment, and comorbidities with anxiety disorders. PTSD and anxiety-related disorders are heterogeneous, which is reflected by the neural circuits involved in the genesis of symptoms that may vary across symptom domains. Treatment is likely to benefit from consideration of this heterogeneity. Continuing efforts are being made to understand the cause of anxiety or stress, the physiological pathways, and the mechanisms which may be utilized to reduce or eliminate anxiety or stress.

The vestibular system may be one pathway to regulating anxiety- and stress-related disorders. The vestibular system is a major contributor to our sense of balance and spatial orientation and consists in each inner ear of three semicircular canals (which detect rotational movement) and the two otolith organs, termed the utricle and saccule, which detect linear acceleration and gravity (Khan & Chang, 2013). They are called otolith organs as they are fluid filled sacs containing numerous free moving calcium carbonate crystals-called otoliths-which move under the influence of gravity or linear acceleration to act upon receptor cells to alter vestibular afferent nerve activity.

The vestibular nuclei, in particular, the medial vestibular nucleus or “MVe”, are located in the pons and medulla and receive input via the vestibular nerve from the vestibular system. The MVe are thought to project (both directly and indirectly via the parieto-insular vestibular cortex (PIVC)) to the brainstem homeostatic sites of the parabrachial nucleus (PB) and the peri-aqueductal gray (PAG) (see Chapter 1 and Chapter 3, Section 8 in doctoral thesis by McGeoch, 2010). The PB seems to act to maintain homeostasis—i.e., a stable internal physiological milieu—by integrating this vestibular input with sympathetic input (via lamina 1 spino- and trigemino-thalamic tract fibers) and parasympathetic input (via the nucleus of the solitary tract) (Balaban and Yates, 2004; Craig, 2007; Craig, 2009; McGeoch et al., 2008, 2009; McGeoch, 2010).

It is thought that the PB then acts to maintain homeostasis by means of behavioral, neuroendocrine, and autonomic nervous system efferent (i.e., both sympathetic and parasympathetic) responses (Balaban and Yates, 2004; McGeoch, 2010). Anatomically the PB projects to the insula and anterior cingulate, amygdala and hypothalamus. The insula and anterior cingulate are areas of cerebral cortex implicated in emotional affect and motivation, and hence behavior (Craig, 2009). The hypothalamus plays a vital role in coordinating the neuroendocrine system (Balaban and Yates, 2004; Fuller et al., 2004; Craig, 2007). The amygdala (together again with the hypothalamus and insula) is similarly known to be important in autonomic nervous system control. The PB also outputs to the PAG and basal forebrain, which are also involved in homeostasis (Balaban and Yates, 2004).

Vestibular nerve stimulation (“VeNS”) activates all five components of the vestibular apparatus simultaneously using an electrical current (Fitzpatrick & Day, 2004; St. George & Fitzpatrick, 2011) and offers the practical option of being produced commercially for home use without expert supervision. VeNS involves stimulating the vestibular system through the transcutaneous application of a small electric current (usually between 0.1 to 3 milliamps (mA)) via two electrodes. The electrodes can be applied to a variety of locations around the head, but typically one is applied to the skin over each mastoid process, i.e., behind each ear. Some authors term this a “binaural application.” If a cathode and an anode are used with one placed over each mastoid, which is the most common iteration, then this is termed a bipolar binaural application of VeNS. The current can be delivered in a variety of ways, including a constant state, in square waves, a sinusoidal (alternating current) pattern and as a pulse train (Petersen et al., 1994; Carter & Ray, 2007; Fitzpatrick & Day, 2004; St. George & Fitzpatrick, 2011).

There have been limited efforts to treat anxiety using vestibular stimulation, however, none have produced any definitive effects or determined useful treatment options. Therefore, there is a need for further development of methods and devices to more effectively and efficiently provide vestibular stimulation to treat anxiety- and stress-related disorders.

### SUMMARY

Embodiments described herein provide for systems, devices and methods for utilizing vestibular stimulation to treat anxiety- and stress-related disorders including PTSD by influencing key areas of the brain and autonomic nervous system responsible for regulating the biochemicals related to anxiety and stress. In some embodiments, stimulation can be delivered for a specified period of time prior to, during or immediately after an anxiety-causing event using customized signal shapes and durations delivered to the vestibular nerves via one or more head-mounted portable electronic devices. In other embodiments, the stimulation can be regularly delivered for a fixed period of time, e.g., 30 to 60 minutes, on a daily basis for an extended period, e.g., weeks or months. An extended treatment period could run indefinitely (months or years) as a maintenance regimen, or until the subject or the supervising medical professional determines that no further improvement is being achieved. The stimulation essentially allows the brain to better respond to an anxiety or stress promoting event, providing an effective method for treating anxiety.

In one embodiment, a method of reducing anxiety and stress in a human subject through delivery of vestibular nerve stimulation (VeNS) comprises: positioning at least one electrode into electrical contact with the human subject and proximate to a location of the subject's vestibular system; and delivering VeNS to the human subject from a current source connected with the at least one electrode.

In another embodiment, a device for reducing anxiety in a human subject comprises electrodes disposed in electrical contact with the subject's scalp at a location corresponding to the subject's vestibular system; and a current source in electrical communication with the electrodes for delivering vestibular nerve stimulation (VeNS) to the subject.

In a further embodiment, a method of treating anxiety with vestibular nerve stimulation comprises positioning at least one electrode into electrical contact with the human subject and proximate to a location of the subject's vestibular system; and delivering VeNS to the human subject from a current source connected with the at least one electrode, wherein the VeNS is delivered for approximately 30 to approximately 60 minutes on a regular basis, e.g., daily, or before, during or after an anxiety event.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is a schematic diagram of an exemplary stimulator circuit for a vestibular nerve stimulation (VeNS) device, according to one embodiment of the invention;

FIG. 2 is a schematic diagram of an alternative embodiment of the stimulator circuit with a gain control component, according to one embodiment of the invention;

FIG. 3 is a schematic diagram of a second alternative embodiment of the stimulator device, according to one embodiment of the invention;

FIGS. 4A and 4B illustrate exemplary wave forms generated by the device, according to one embodiment of the invention;

FIG. 5 is a diagram showing an exemplary VeNS electrode placement, according to one embodiment of the invention;

FIG. 6 is a diagram illustrating the vestibular system of the left inner ear;

FIG. 7 is a model illustrating anatomical features linking the vestibular and circadian timing systems (CTS);

FIG. 8 is a diagram illustrating an exemplary wave form for use in delivering VeNS, according to one embodiment of the invention;

FIG. 9 is a flow diagram illustrating an example method for utilizing VeNS to treat anxiety, according to an embodiment of the invention;

FIG. 10A is an illustration of a vestibular nerve stimulation device positioned around a mastoid process and an ear of a patient for use in treating anxiety, according to one embodiment of the invention;

FIG. 10B is an illustration of a circular flow of current between an anode and a cathode provided by the vestibular nerve stimulation device, according to one embodiment of the invention;

FIG. 10C is an illustration of a wireless communication capability between vestibular nerve stimulation devices positioned on opposing ears of the patient, according to one embodiment of the invention;

FIG. 10D is an illustration of the vestibular nerve stimulation device incorporated into an around-ear headphone, according to one embodiment of the invention;

FIG. 11 is a diagrammatic view of a head-mounted device set for delivery of VeNS according to an embodiment of the invention.

FIG. 12 is a block diagram illustrating an example wired or wireless processor enabled device that may be used in connection with various embodiments described herein;

FIG. 13 is a table with results of a trial evaluating efficacy of VeNS treatment for PTSD.

FIG. 14 is a plot comparing PCL-5 scores for subjects treated using the inventive device and method versus a control group.

### DETAILED DESCRIPTION OF EMBODIMENTS

Certain embodiments disclosed herein provide for stimulation of the vestibular system in such a way as to reduce the physiological reactions of the autonomic nervous system and reduce anxiety and stress in a subject. For example, one method disclosed herein allows for a device with one or more electrodes placed over a subject's scalp to deliver vestibular nerve stimulation (VeNS) to the vestibular nerve, which is then carried into the vestibular nucleus in the brainstem and thereafter transmitted to the neurological components of the autonomic nervous system to affect areas which trigger an anxiety response, allowing the body reduce or prevent an anxiety and/or stress response. For purposes of the present disclosure, disorders may be described interchangeably as “anxiety-related,” “stress-related,” or “stressor-related.” Each phrase includes, but is not limited to, generalized anxiety disorder (GAD) and trauma/stressor-related disorders including post-traumatic stress disorder (PTSD). The inventive approaches disclosed herein are intended to be applicable to such disorders. The characteristics of the stimulation signal and duration of the treatment are configured to allow the treatment to be delivered on a regular basis, such as daily, or before, during or after an anxiety or stress event that might otherwise trigger an anxiety or stress response in the subject.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### Vestibular Stimulation Devices

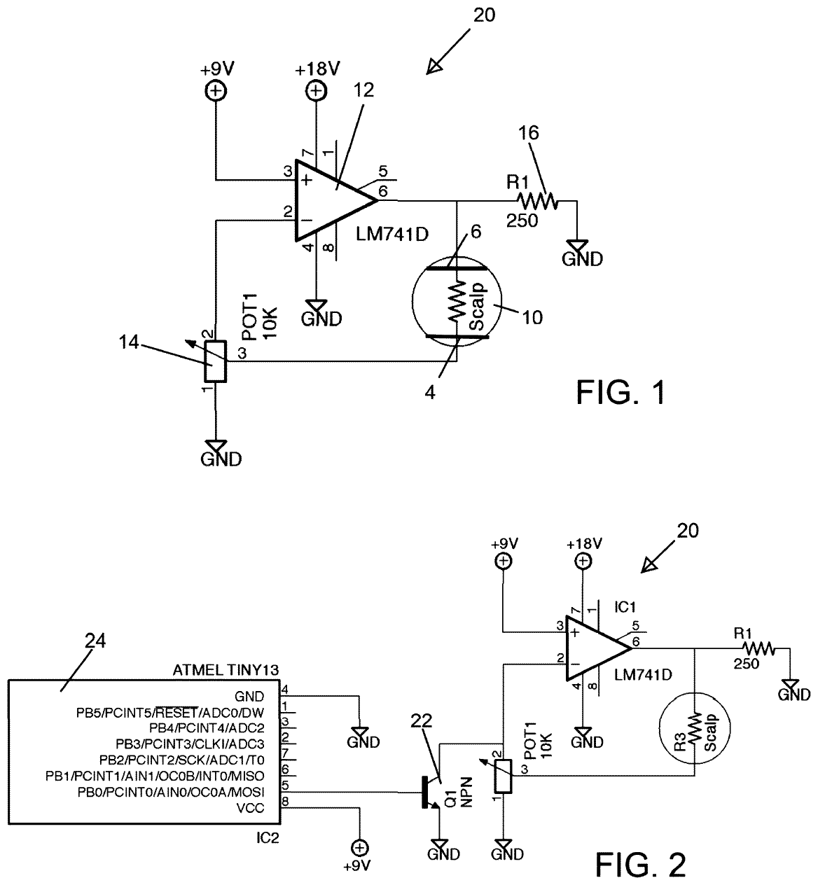

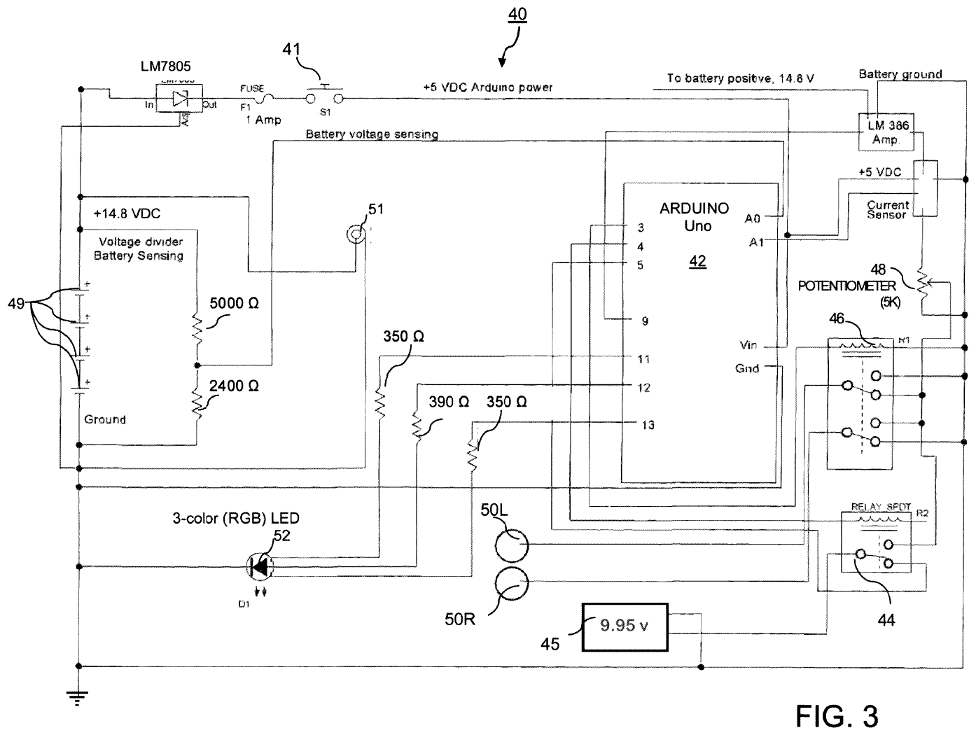

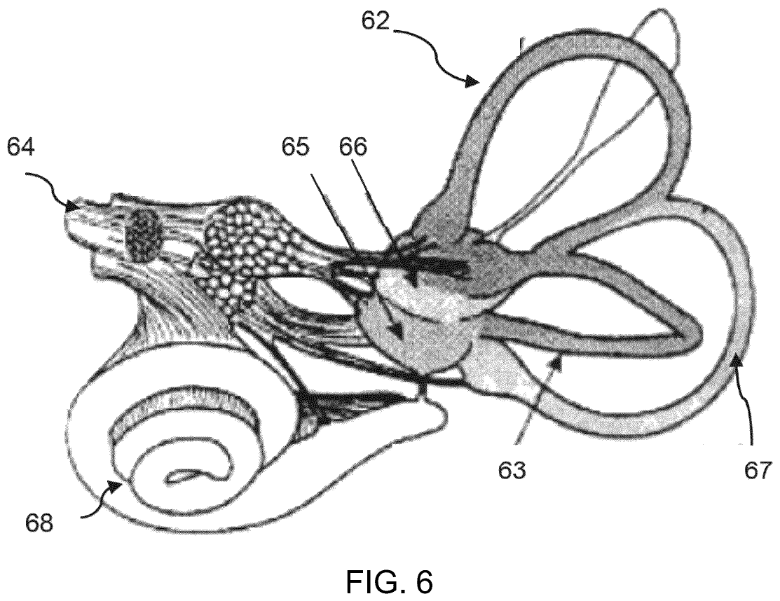

FIGS. 1 and 2 illustrate one possible embodiment of the VeNS circuitry that can be employed to carry out the method of the present invention. The device 20 includes a source of time-varying galvanic current that may be software programmable using a microcontroller. In one embodiment, vestibular stimulation may be provided via a head-mounted portable electronic device which is comfortably positioned onto a user's head in an area where stimulation can be delivered to one or both sides of the user's vestibular nerves. Additional illustrations of devices are provided in FIGS. 5, 10A-10D and 11.

FIG. 1 illustrates the basic components of an embodiment of the stimulation device 20, which includes an operational-amplifier (“op-amp”) based constant-current source. A voltage is placed across the scalp 10 through electrodes 4 and 6 and measured by the op-amp 12. In the exemplary embodiment, op-amp 12 may be a general purpose operational amplifier, an example of which is the LM741 series op-amp, which is widely commercially available. Selection of an appropriate operational amplifier will be within the level of skill in the art. If the voltage returning from the scalp 10 to pin 2 (inverting input) of op-amp 12 is different than the reference voltage +9V at pin 3 (non-inverting input), the operational amplifier draws from the +18V input through pin 7 to increase the amount of voltage output at pin 6, thereby increasing the current across the scalp 10 to maintain a constant current level. Load resistor 16 is 250 ohms. Adjustment of potentiometer 14 provides gain control by decreasing the voltage input into op-amp 12 at pin 2, thus controlling the amount of current flowing across the scalp. In the preferred embodiment, the +9V and +18V inputs are provided by one or more batteries (not shown), or a conventional DC converter may be used with appropriate safety provisions.

The schematic in FIG. 2 adds control components to the basic stimulator circuit 20 of FIG. 1. Transistor 22, powered by the pulse-width-modulation (PWM) output (MOSI (master output/slave input, pin 5) of an ATtiny13 microcontroller 24 (Atmel Corporation, San Jose, CA) or similar device, may be used to control the gain of the stimulator. The PWM causes the transistor to draw more or less of the voltage entering the Op-Amp 12 (pin 2) to ground, thus modulating the amount of current flowing across the scalp.

In a preferred embodiment, the device components and any external interfaces will be enclosed within a housing 30 (shown in FIG. 5) with appropriate user controls 32 for selecting stimulation parameters as appropriate. Note that a knob is shown for illustrative purposes only and that other types of controls, including switches, buttons, pressure bumps, slides, touch screens or other interface devices may be used. Optional design components that may be added to expand the functionality of the device include a memory storage device, such as a memory card or electrically erasable programmable read-only memory (EEPROM), which will allow the time, duration, and intensity of stimulations to be recorded. This can be accomplished by programming the microcontroller 24 to output a logic-level 3.4V pulse (TTL (transistor-transistor logic)) from the remaining digital out (MISO (master input/slave output, pin 6) to a secure digital (SD) memory card, EEPROM, USB flash drive or other data storage device via an appropriate port on the device housing. Additionally, the +18V input may be derived by integrating a charge pump, or DC-DC step-up converter, such as the MAX629 or MAX1683 (not shown). This design feature would have the benefit of reducing the size of the device by producing the necessary +18V input from smaller batteries, which can be disposable or lithium ion rechargeable. Additional features may include wireless communication circuitry, as is known in the art, for programming and/or data collection from a remote computing device, which may include a personal computer, smart phone or tablet computer.

Other functions for implementing VeNS in the present invention may include the ability to pulse the current at precise intervals and durations, in a sinusoidal wave with adjustable amplitude and period, and even switch polarity at precise intervals.

Additional options for facilitating and/or enhancing the administration of VeNS may include a built-in biofeedback capability to adjust the stimulation parameters for optimal effect based on signals generated by sensors that monitor the subject's activity and/or biometric characteristics, such as motion, position, heart rate, etc. For example, real-time heart measured by a heart-rate sensor or monitor can be used as input into the VeNS device, triggering an automatic adjustment of the sinusoidal VeNS frequency to an appropriate, possibly pre-programmed, fraction of the cardiac frequency. Real-time data on the user's motion or position measured by accelerometers may also be used as input to control stimulation, to improve effectiveness and safety. For example, treatment could be terminated if excessive motion or change in the user's position is detected, or the user can be alerted about changes in position that could have adverse effects. The heart rate sensor/monitor and/or accelerometers may be separate devices that communicate with the inventive VeNS device through a wired or wireless connection. Alternatively, sensors may be incorporated directly into the VeNS device to form a wearable “sense-and-treat” system. As new sensors are developed and adapted to mobile computing technologies for “smart”, wearable mobile health devices, a “sense-and-treat” VeNS device may provide closely tailored stimulation based on a wide array of sensor data input into the device.

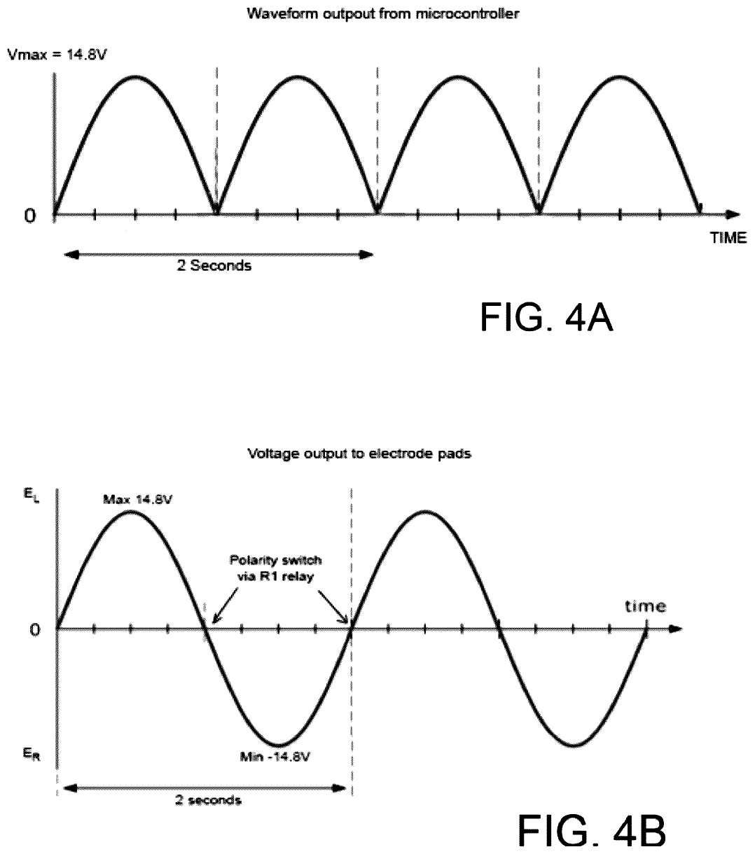

FIG. 3 schematically illustrates an exemplary prototype of the inventive device 40 implemented using the widely commercially-available ARDUINO® Uno single board microcontroller 42 (Arduino, LLC, Cambridge, MA), which is based on the ATmega328 microcontroller (ATMEL® Corporation, San Jose, CA). Microcontroller 42 includes fourteen digital input/output pins (of which six can be used as pulse width modulation (PWM) outputs), six analog inputs, a 16 MHz ceramic resonator, a USB connection, a power jack, an ICSP header, and a reset button. The +14.8 V DC power to the circuit is provided by batteries 49. For example, four lithium ion batteries, each providing 3.7V (1300 mAh) are used, and are preferably rechargeable via charging port 51.

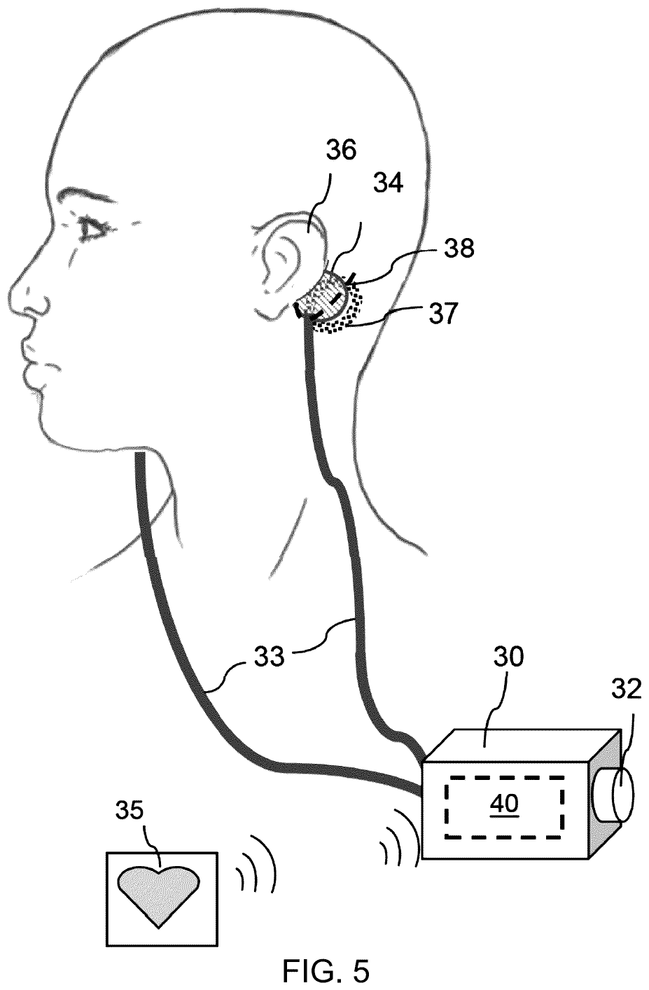

The PWM allows the output waveform to be accurately controlled. In this case, the waveform takes a repeating half-sine wave pattern in a positive deflection, as shown in FIG. 4A. The frequency has been predefined as 0.25 Hz but may be set to a different value by manual control or in response to input from a sensor, such as a heart rate sensor (see, e.g., FIG. 5). The user can manually control the amplitude by adjusting the potentiometer 48, allowing a range of 0 to 14.8V to be supplied to the electrodes. This adjustment may be effected by rotating a knob, moving a slide (physically or via a touch screen), or any other known user control mechanism. Alternatively, the potentiometer setting can automatically adjust in response to an input signal from a sensor. Relay 44 communicates the voltage adjustment to a graphical display 45 to provide a read-out of the selected voltage and/or current.

A relay 46 may be employed to effectively reverse the polarity of the current with every second pulse. The effect of this is shown in FIG. 4B, where the sinusoidal pattern changes polarity, thus generating a complete sine waveform to produce alternating periods of stimulation, on the order of 1 second in duration, to the left and right mastoid electrodes 50L and 50R.

The device may optionally include a three color LED 52 that provides a visual display of device conditions, i.e., diagnostic guidance, such as an indication that the device is working correctly or that the battery requires recharging.

Optional design components may include a touch screen configuration that incorporates the potentiometer controls, a digital display of voltage and current, plus other operational parameters and/or usage history. For example, remaining battery charge, previous stimulation statistics and variations in resistance could be displayed. Additional features may include controls for alterations in the waveform such as change of frequency and change of wave type (for example square, pulse or random noise). The ARDUINO® microprocessor platform (or any similar platform) is ideally suited to incorporate feedback control or manual control of frequency, intensity or other stimulation parameters based on an external signal source. For example, the ARDUINO® microprocessor platform, if provided with BLUETOOTH® capability, can be wirelessly controlled by an iPHONE®, ANDROID®, or other smart phone, laptop or personal computer, tablet or mobile device, so that the touchscreen of the mobile device can be used to control and/or display the VeNS stimulation parameters rather than requiring a dedicated screen on the device. The mobile device may also be configured to store and analyze data from previous stimulations, providing trends and statistics about long periods of stimulation, such as over 6 months. Applications of this could allow for programs to monitor and guide users on their progress and goals, highlighting body measurements and changes in weight relative to the periods of stimulation.

An exemplary operational sequence for the embodiment of FIG. 3 for use in treating anxiety may include the following steps:

When the push button power switch 41 is activated, the battery(ies) 49 supply 5 volts DC to the microprocessor 42 through a 5 volt regulator and a 1 amp fuse (shown in the figure but not separately labeled.)

The LED 52 will flash green three times to indicate the power is “on”. If the blue light flashes the battery needs charging. While the voltage is supplied to the electrodes 50L and 50R, the LED 52 will flash red at regular intervals, e.g., 30 seconds to a minute.

The microprocessor 42 generates a 0.75 VDC half wave sign wave. The voltage is amplified to 14.8 volts by the amplifier. The sine wave completes one-half cycle in 1 second (i.e., the frequency of the sine wave is 0.25 Hz). The voltage can be varied by the potentiometer 48 from 0 to 14.8 volts.

After a half cycle is completed, relay 46 switches polarity of the electrodes 50L, 50R and the microprocessor 42 sends another half cycle. The relay 46 again switches polarity and continues for as long as the unit is “on”. This sends a full sine wave of up to +14.8 VDC to the electrodes, with the full voltage swing modulated by the potentiometer 48.

A digital display 45 provides a visual indication of the voltage and current delivered to the electrodes 50L, 50R. Depending on the size and complexity of the display, voltage and current values may be displayed simultaneously or alternately for a short duration, e.g., 3 seconds.

Other device options may include user controls to allow the current to be pulsed at precise intervals and durations, a sinusoidal wave to be generated with adjustable amplitude and period, and/or to switch polarity at precise intervals. External control and monitoring via a smart phone or other mobile device as described above may also be included. Further input and processing capability for interfacing and feedback control through external or internal sensors may be included.

FIG. 5 illustrates an exemplary VeNS electrode 34 positioned on the skin behind the pinna of the left ear 36, and over the left mastoid process, of a subject to be treated. The mastoid process is represented by dashed line 38. The right electrode (not shown) would be placed in the same manner on the skin over the right mastoid process and behind the right pinna. It should be noted that the illustrated placement of the electrodes is provided as an example only. In fact, laterality of the electrode application, e.g., electrodes precisely over both mastoid processes, is not believed to be critical, as long as each electrode is in sufficient proximity to the vestibular system to apply the desired stimulation. The electrodes 34 are connected to stimulation device 40 (inside housing 30) by leads 33. Manual control means, illustrated here as a simple knob 32, may be operated to control the current or other parameters. As described above, alternative control means include a slide, touch screen, buttons or other conventional control devices. External control signals, for example, a signal from a heart rate monitor 35, may be input into the device either wirelessly, as illustrated, or by leads running between the sensor and the device. Electrodes such as the widely commercially available 2×2 inch platinum electrodes used for transcutaneous electrical nerve stimulation (TENS) may be used in order to minimize any possible skin irritation. A conducting gel 37 may be applied between the subject's scalp and the contact surface of the electrodes to enhance conduction and reduce the risk of skin irritation.

The amount of current the subject actually receives depends on the scalp resistance (I_{scalp}=V_{electrodes}/R_{scalp}), which may vary as the user perspires, if the electrode position changes, or if contact with the skin is partially lost. It appears that the current levels quoted in the literature could only be delivered if the scalp resistance was much lower than it actually is. Measurements conducted in conjunction with the development of the inventive method and device indicate that the trans-mastoid resistance is typically between 200 to 500 k-Ohm. Thus, if a VeNS device were actually being used to deliver 1 mA, the voltage would be between 200 to 500V according to Ohm's law. The battery-powered devices that are usually used to administer VeNS are simply not capable of generating such an output. Hence, the existing reports appear to be inaccurate with regard to the actual current being delivered in VeNS.

Prior art designs lack consideration for each subject's unique scalp resistance and therefore may not deliver an effective current to each patient. In the present invention, this limitation can be overcome by taking into account inter-subject scalp resistance variability as well as compensating for fluctuations in the scalp resistance that may occur throughout the procedure. To compensate for slight and fluctuating changes in scalp resistance during the administration of current, the inventive VeNS device may include an internal feedback loop that continuously compares the desired current against the actual measured current across the scalp and automatically compensates for any differences. If R_{scalp} increases, the V_{electrodes} increases to compensate. Conversely, voltage decreases when R_{scalp} drops. This dynamic feedback compensation loop provides constant current across the scalp for the duration of the procedure regardless of fluctuating changes in electrode-scalp impedance.

### Pathways for Anxiety Therapy

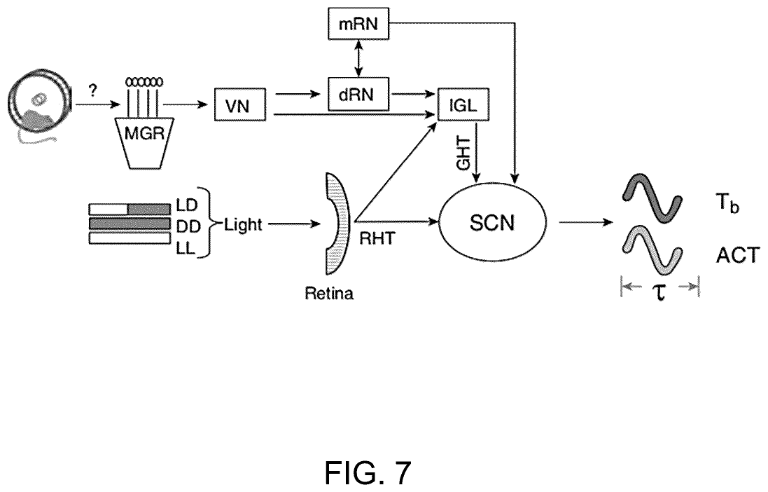

FIG. 6 illustrates the vestibular system of the left inner ear. The cochlea 68, which is the peripheral organ of hearing, is also shown. It demonstrates: the anterior 62, posterior 67, and horizontal 63 semicircular canals, which transduce rotational movements; and the otolith organs (the utricle 66 and saccule 65), which transduce linear acceleration and gravity. The vestibulocochlear nerve 64 (also known as the eighth cranial nerve) is composed of the cochlear nerve (which carries signals from the cochlea), and the vestibular nerve (which carries signals from the vestibular system).

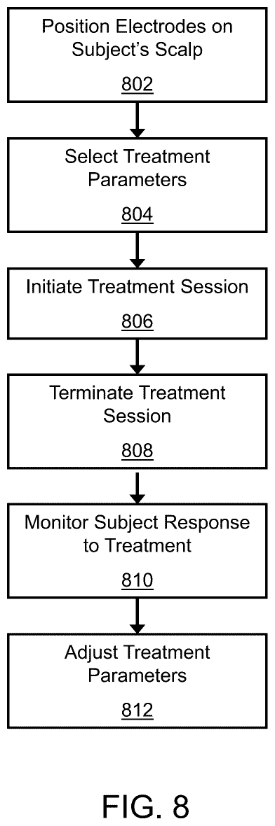

Misalignment of circadian rhythm has been shown to provoke anxiety and other mood disorders. FIG. 7 is a model outlining potential anatomical features linking the vestibular and circadian timing systems (CTS). Light, the primary synchronizing agent for the CTS, is transmitted to the suprachiasmatic nucleus (SCN) via the retinohypothalamic tract (RHT). Nonphotic stimuli, such as locomotor activity (running wheel), are transmitted to the SCN via the intergeniculate leaflet (IGL) and the geniculohypothalamic tract (GHT). There is also evidence supporting involvement of the serotonergic midbrain raphe (dorsal and medial, dRN and mRN, respectively) in the transmission of activity information to the SCN and IGL. Morphological data also suggest that the vestibular nuclei (VN) may influence the raphe nuclei, particularly the dRN. MGR are the macular gravity receptors, T is the circadian period, and Tb is body temperature.

Vestibular stimulation activates key areas of the brain related to anxiety indirectly by using the vestibular nucleus as a relay, transmitting stimulation of the vestibular system from the vestibular nucleus to the SCN, IGL and hypothalamus. These neurological components influence the physiological response to an anxiety inducing event in the human body, so the application of VeNS essentially re-regulates this physiological response and reduces an anxiety level in the subject.

### Treatment Methods

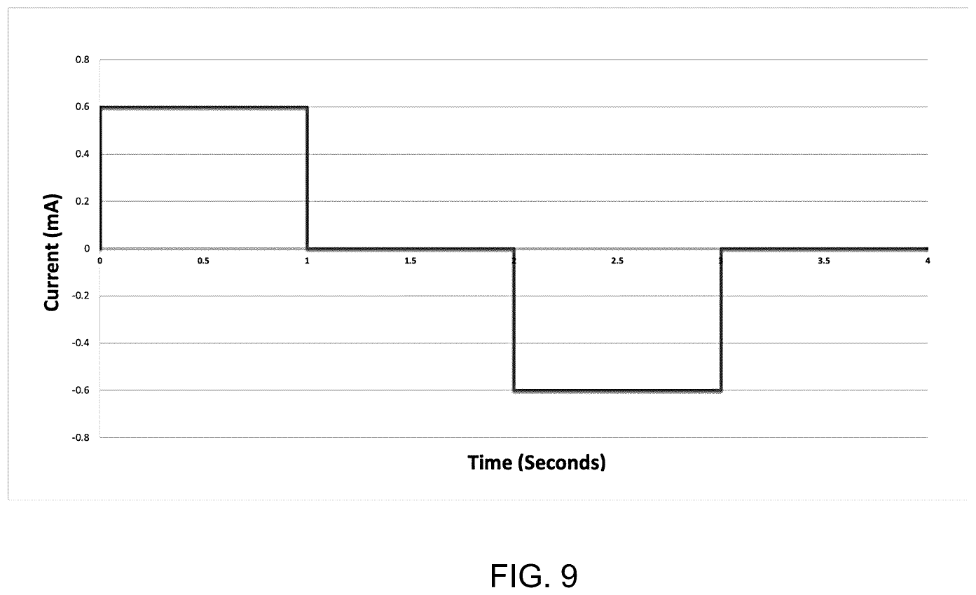

FIG. 8 illustrates one embodiment of a method of utilizing VeNS to reduce anxiety in a human subject. In step 802, one or more electrodes are positioned on the subject's scalp proximate to the location of the vestibular system. The electrodes may be placed on one or both sides of the scalp near the approximate location where stimulation of the vestibular nerve can be achieved. In step 804, the parameters of the VeNS treatment are configured on the VeNS device depending on one or more factors relating to the treatment or the subject, such as the signal shape, pulse, frequency, duration of treatment, time before or after an anxiety event, etc. Once the parameters are selected, the treatment session may be initiated in step 806. In step 808, at the end of the desired treatment duration, the treatment is terminated. In step 810, the subject's response to the treatment may be monitored to determine the effectiveness of the treatment, for example via remote or wearable sensors, the subject's own observations about their anxiety level and duration, and other physiological and psychological factors that may be measured over longer periods of time after multiple treatment sessions. In step 812, the subject's response to the treatment may be utilized to adjust the overall treatment schedule, the parameters of the VeNS or other observed factors that may be influencing the subject's anxiety.

The method of treatment may include delivery of vestibular stimulation at a range of frequencies that are effective at re-regulating autonomic nervous system. In one embodiment, the parameters of a VeNS treatment includes use of a square wave with a frequency of approximately 0.25 Hz and a current range of approximately 0.01 mA-1.5 mA delivered at an approximately 50 percent duty cycle. The electrodes may be placed bilaterally for delivery of stimulation to both sides of the user's head. The session length of treatment may be approximately 30 minutes to approximately 60 minutes.

In another embodiment, the method of treatment may include delivery of vestibular stimulation at varying parameters that may be effective for different types of subjects or with different outcomes relating to the timing of the treatment and the level of anxiety. For example, a range of frequencies from approximately 0.0001 Hz to approximately 10000 Hz, with a range of approximately 0.01 mA to approximately 5 mA, may be utilized with any type of waveform and duty cycle, from square to sinusoidal to pulse. The treatment may be delivered via only one electrode placed on one side of the user's head at the approximate location where stimulation of the vestibular nerve can be made. The user may initiate a treatment at any time, either on a regular basis (daily), or prior to, during, or after an anxiety event and initiate a treatment session of predetermined duration, which may range from approximately 1 minute (e.g., for acute events) to approximately 120 minutes. Typically, a treatment session will be on the order of 30 minutes to 60 minutes.

In addition to treatments of anxiety and anxiety-related disorders, the aforementioned methods may also be useful in treating other mental health disorders with similar physiological pathways, such as schizophrenia.

### Devices for Delivering Treatment of Anxiety- and Stress-Related Disorders

A comparable commercially available VeNS device sold under the trademark VESTIBULATOR™ (Good Vibrations Engineering Ltd. of Ontario, Canada) has previously been used in a number of research studies at other institutions. (Barnett-Cowan & Harris, 2009; Trainor et al., 2009.) This device functions with 8 AA batteries, so that the voltage can never exceed 12 V. According to the manufacturer's specifications, the maximum current that this device can deliver is 2.5 mA. The present invention uses a more user-friendly device (e.g., the delivered current can be adjusted using a controller (knob, slide, or similar) on the side of the housing, in comparison to the VESTIBULATOR™, where a similar adjustment can only be carried out by first writing a MATLAB® script and then uploading it remotely, via BLUETOOTH®, in order to reprogram the VESTIBULATOR's™ settings.)

Due to the very small currents used during VeNS, the technique is believed to be safe (Fitzpatrick & Day, 2004; Hanson, 2009). In particular, although electrical current can lead to cardiac arrhythmias, including ventricular fibrillation, the threshold for such an occurrence is in the 75 to 400 mA range, well above the current levels the battery powered VeNS devices can deliver. Furthermore, the electrodes will only be applied to the scalp, such as shown in FIG. 5, and nowhere near the skin over the chest.

Resistive heating can occur with high voltage electrical stimulation of the skin. However, the voltage and current (usually below 1 mA) delivered during VeNS are well below the levels that pose this risk. Nonetheless, skin irritation can occur due to changes in pH. This may be mitigated by using large surface area (approximately 2 inch diameter) platinum electrodes and aloe vera conducting gels.

It may be desirable to monitor the subject's heart rate (HR) to determine the cardiac frequency during VeNS treatment. The cardiac frequency can then be used to alter the frequency of the sinusoidal VeNS so as to maintain a certain ratio between the cardiac frequency and the frequency of the sinusoidal VeNS to avoid interference with baroreceptor activity. For example, a sinusoidal VeNS frequency to cardiac frequency ratio of 0.5 would be appropriate.

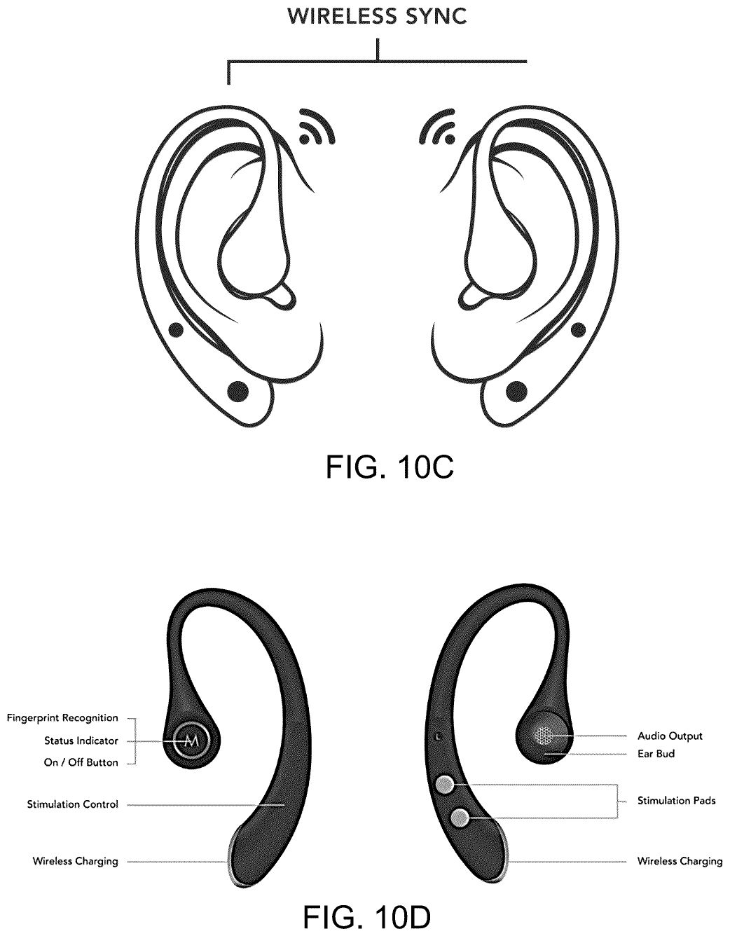

During administration of VeNS, one platinum electrode is attached to the skin over one mastoid and the other electrode attached to the skin over the other, as shown in FIG. 5. The electrodes may be coated with conducting gel containing aloe vera. The device is activated to deliver a current of approximately 0.1 mA (given a trans-mastoid resistance of about 500 kOhm) with a sinusoidal or other alternating polarity waveform at approximately 0.25 Hz. A typical current range for the device would be around 0.001 mA to around 5 mA, with a preferred range being 1 mA to 1.5 mA. The subject may need to remain seated or lying flat throughout the session to avoid the effects of altered balance during vestibular stimulation, although the device described in FIGS. 10A-10D would not be limited as such. The device may be set up to automatically stop after one hour, however, the subject may discontinue the treatment sooner if desired. If their balance has been altered, the subject should remain seated until their balance has returned to normal, which should occur within a short period of time after the VeNS device has been turned off.

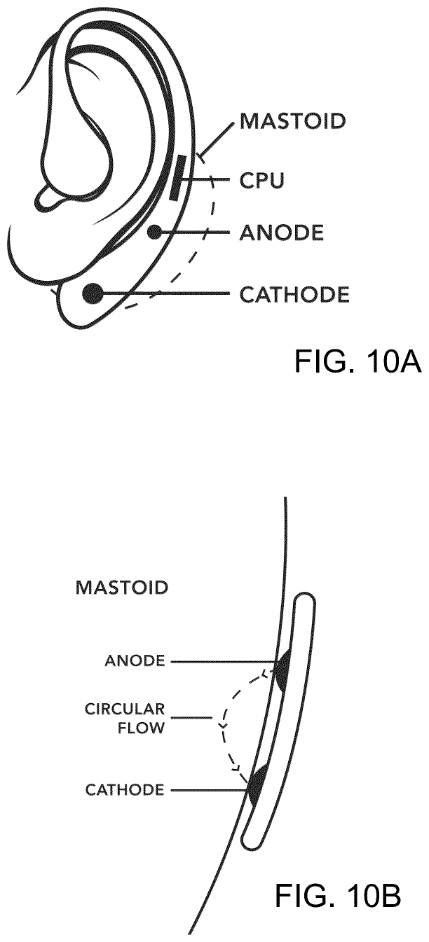

In one embodiment, a VeNS device provided by the company Neurovalens Limited was used to deliver the stimulation. This device delivers a VeNS current waveform as illustrated in FIG. 9, which consists of an AC square wave at approximately 100 Hz with an approximately 50% duty cycle. The protocol followed was that for the first 30 minutes each subject underwent indirect calorimetry alone in order to establish a baseline. Each subject then underwent a one-hour session of binaural, bipolar VeNS with electrodes placed on the skin over each mastoid as shown in FIG. 5. As stated above, an AC square wave at approximately 100 Hz with an approximately 50% duty cycle was delivered, in all subjects with a current of 0.6 mA, although the device used is capable of delivering more.

FIG. 10A illustrates one possible embodiment of a VeNS stimulator which avoids the side-effect of altering a balance of the subject during stimulation by providing simultaneous binaural stimulation from two devices secured on both mastoid processes. Specifically, two stimulators are configured for placement around the subject's left and right ears such that the anode and cathode electrodes are in electrical contact with each mastoid process on the right and left side of the subject's head. The device includes a source of time-varying galvanic current that may be software programmable using a microcontroller (CPU), including a communications interface which allows the VeNS stimulator to communicate with one or more additional stimulators or a master controller in order to synchronize multiple stimulators and coordinate the delivery of VeNS to various locations where the vestibular system may be affected. In one embodiment, vestibular stimulation may be provided via this ear-mounted portable electronic device which is comfortably positioned over a subject's ear in an area where stimulation can be delivered to one or both sides of the user's vestibular nerves.

FIG. 10B is a side view illustration of the VeNS stimulator which illustrates how the stimulator is a single-sided two pad device with the anode and cathode pads positioned on the same side of the stimulator and adjacent with the mastoid process of the subject. FIG. 10B additionally illustrates how the flow of current in the VeNS stimulator moves from the anode to the cathode across the mastoid process in order to stimulate the vestibular system. While some vestibular stimulation devices place an anode on one stimulation pad on one side of the subject's head and a cathode on a second stimulation pad on the other side of the subject's head in order to deliver the stimulation across the subject's brain, this VeNS stimulator provides both the anode and cathode on a single stimulation pad and thus creates a localized stimulation of the vestibular nerve. This localized stimulation can then be replicated at another location, as will be described immediately below.

FIG. 10C illustrates one embodiment of a set of binaural, synchronized VeNS stimulators, with a first stimulator located on the left ear of the subject and a second stimulator located on the right ear of the subject. The two VeNS stimulators may communicate wirelessly in order to synchronize and coordinate the delivery of VeNS to the subject from both locations. In one embodiment, the VeNS stimulators deliver VeNS simultaneously from their respective locations such that the left vestibular nerve and right vestibular nerve receive stimulation at the same time. By delivering the treatment simultaneously from opposite sides of the subject's head, the rocking sensation that can occur from delivering treatment on only one side of the subject's head (or from delivering stimulation from an anode on one side of the head to a cathode on the other side of the head) is avoided.

In another embodiment, the portion of the VeNS stimulator which fits within the ear canal may also have a set of pads to provide additional stimulation through the ear canal.

Furthermore, in one embodiment the VeNS stimulator may be incorporated into a portable electronic device such as an around-ear headphone, as illustrated in FIG. 10D. The VeNS stimulator may include an ear bud portion with a speaker which fits into the ear canal. The ear bud portion may also include a power button, LED status indicator and a fingerprint recognition sensor for identity management. A lower portion of the VeNS stimulator may be configured for wireless charging.

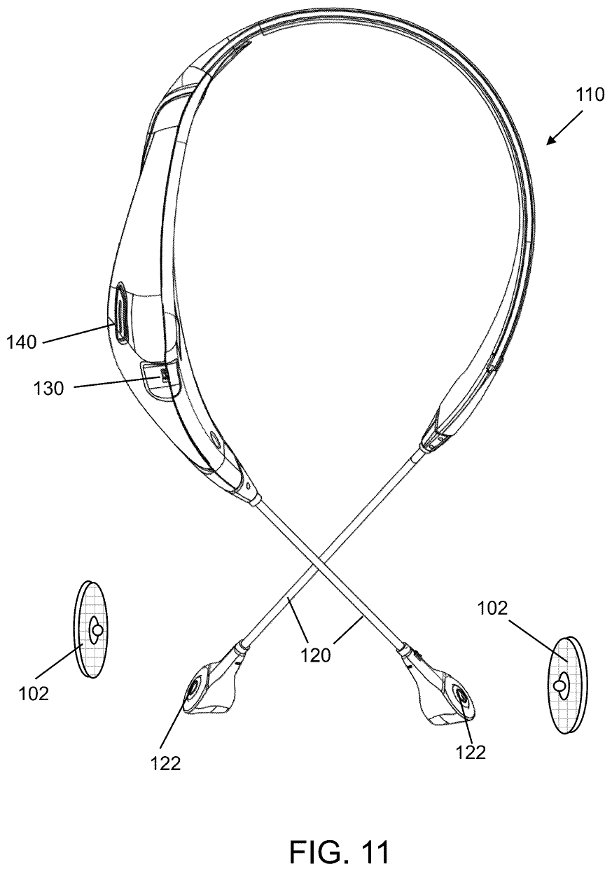

FIG. 11 illustrates an exemplary embodiment of a head worn device that may be employed for administering VeNS to the subject. For use, to ensure good contact, the area behind the ear lobe may be cleaned using an alcohol wipe. A removable (disposable) adhesive electrode pad 102 with a snap fastener is then affixed just behind the subject's ear near the mastoid process on each side. (Such pads are widely commercially available.) The headset 110 is placed over the subject's head to rest comfortably, like a headband, on top of the head. The flexible cables 120 extending from headset 110 include fasteners 122 that releasably mate with the fastener on electrode pads 102. Once in place, the user can turn on the device by depressing power button 130. LED indicator 140 illuminates to indicate activation. The power button 130 may be used to increase or decrease stimulation by pressing multiple times, or separate buttons (not shown) can be provided to change the stimulation level.

The incorporation of the VeNS stimulator into a consumer electronic device such as a pair of headphones provides an opportunity for the subject to receive anxiety treatment in public-including during an anxiety-causing event-without the stigma of a conspicuous-looking medical device. For example, a subject could wear the device during a social event which causes anxiety in the subject in order to alleviate the subject's anxiety during the event. Additionally, the subject could wear the device prior to attending the social event in order to prevent or reduce an anxiety response. And alternatively, if the subject experiences an unanticipated anxiety response from an event, the device can be worn after the anxiety event to reduce the anxiety response in the subject.

### Computer-Enabled Embodiment

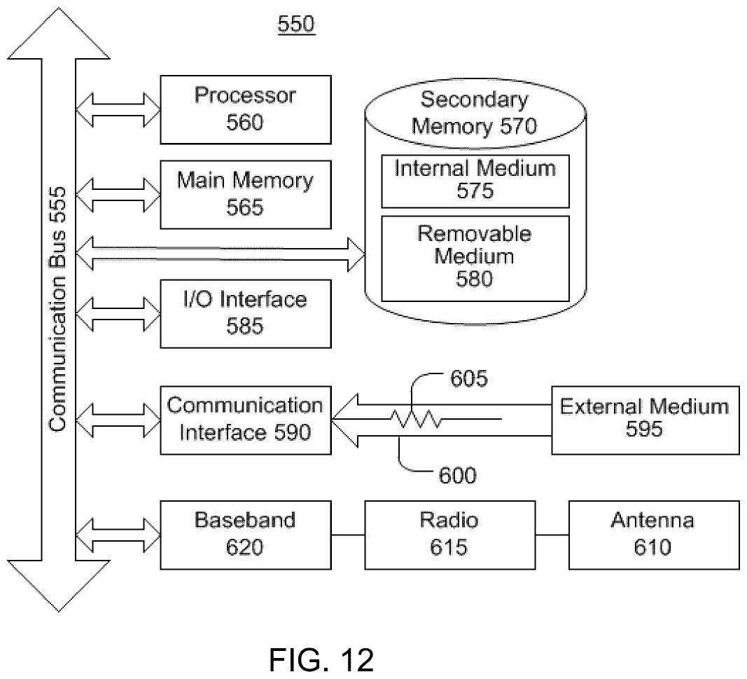

FIG. 12 is a block diagram illustrating an example wired or wireless system 550 that may be used to implement the inventive methods and devices disclosed herein. For example, the system 550 may be used as or in conjunction with a vestibular nerve stimulation device as previously described with respect to FIGS. 1-10. The system 550 can be a conventional personal computer, computer server, personal digital assistant, smart phone, tablet computer, or any other processor-enabled device that is capable of wired or wireless data communication. Other computer systems and/or architectures may be also used, as will be clear to those skilled in the art.

The system 550 may include one or more processors, such as processor 560. The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown).

System 550 preferably includes a main memory 565 and may also include a secondary memory 570. The main memory 565 provides storage of instructions and data for programs executing on the processor 560. The main memory 565 is typically semiconductor-based memory. The secondary memory 570 may optionally include an internal memory 575 and/or a removable medium 580. The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or an external magneto-optical drive.

System 550 may also include an input/output (“I/O”) interface 585. The I/O interface 585 facilitates input from and output to external devices. For example, the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590 configured to implement industry promulgated protocol standards as are known in the art.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. These non-transitory computer readable mediums are means for providing executable code, programming instructions, and software to the system 550.

The system 550 also includes optional wireless communication components that facilitate wireless communication. The wireless communication components comprise an antenna system 610, a radio system 615 and a baseband system 620. In the system 550, radio frequency (“RF”) signals are transmitted and received over the air by the antenna system 610 under the management of the radio system 615.

The central processing unit 560 is configured to execute instructions (i.e., computer programs or software) that can be stored in the memory 565 or the secondary memory 570. Such computer programs, when executed, enable the system 550 to perform the various functions of the present invention as previously described.

Those of skill in the art will appreciate that the embodiments disclosed herein can be implemented as electronic hardware, computer software, or combinations of both.

### Example 1: Treatment of Post Traumatic Stress Disorder (PTSD)

PTSD is evaluated based on criteria of causes and symptoms defined by the American Psychiatric Association in its Diagnostic and Statistical Manual of Mental Disorders (DSM-5). (see, e.g., Blevins et al., The Posttraumatic Stress Disorder Checklist for DSM-5 (PCL-5): Development and Initial Psychometric Evaluation, J Trauma Stress 2015 December; 28 (6): 489-98.) PCL-5 (Posttraumatic Stress Disorder Checklist for DSM-5) is a self-report questionnaire that screens for PTSD and assesses symptom severity. The PCL-5 has a variety of purposes, including monitoring symptom change during and after treatment, screening individuals for PTSD, and making a provisional PTSD diagnosis. The PCL-5 is a 20-item questionnaire, corresponding to the DSM-5 symptom criteria for PTSD. The wording of PCL-5 items reflects both changes to existing symptoms and the addition of new symptoms in DSM-5. The self-report rating scale is 0-4 for each symptom. Rating scale descriptors are the same: “Not at all,” “A little bit,” Moderately,” “Quite a bit,” and “Extremely.” A total symptom severity score (range—0-80) can be obtained by summing the scores for each of the 20 items. A 5 to 10 point change represents reliable change (i.e., change not due to chance) and a 10 to 20 point change represents clinically significant change. Therefore, it has been recommended to use 5 points as a minimum threshold for determining whether an individual has responded to treatment and 10 points as a minimum threshold for determining whether the improvement is clinically meaningful.

The inventive method and device were evaluated for efficacy and safety of VeNS as a method of treatment of PTSD compared to a sham control. The primary objective was to establish the clinical performance of VeNS device stimulation effect in adults with PTSD. For testing, the head worn MODIUS® Spero™ device (similar to the device shown in FIG. 11), manufactured by Neurovalens Limited of Belfast, UK, was used to deliver VeNS stimulation as described above (alternating polarity square wave with 50% duty cycle) at a frequency of 0.25 Hz for a specified period of time, typically around 30 minutes per day. The clinical performance was evaluated in terms of the outcomes of the primary efficacy (reduction in the severity of PTSD symptoms assessed by the PCL-5) and primary safety endpoints (occurrence of device related adverse events) for the device, in comparison to a sham control device.

The sham device is identical in appearance to the active device and interacts with an application for recording and uploading usage data in a similar manner to the app used with the active device. It applies some stimulation to a user for a short period of time (around 30 seconds), before tapering down to zero over a further ˜20 seconds, thus creating the impression in the user of an active device. The device was placed on the head in a manner analogous to headphones with hydrogel electrodes placed over the mastoid processes as described above with reference to FIG. 11. Participants were advised to use the device for 30 minutes per day.

The efficacy analyses were performed on the Intention to Treat (ITT) population, which includes all participants randomized to receive either the VeNS or sham control devices. The adjusted ITT population (ITTa) included participants randomized and have baseline PCL-5 data. Per Protocol (PP) population excludes any participants who violated the protocol in terms of a pre-defined major deviation. The primary efficacy analysis was conducted on each of the ITT, ITTa and PP populations.

The primary efficacy endpoint is the change in the Post-Traumatic Stress Disorder Checklist (PCL-5) score from baseline to 12 weeks between the active and sham groups.

For the primary efficacy endpoint (i.e. change from baseline in PCL-5 at 12 weeks), all available data were used and missing data were handled using multiple imputation. The missing data were imputed multiple times to generate 20 imputed datasets. For the primary efficacy endpoint, imputed values for change from baseline to week 12 in the PCL-5 score was predicted using a linear regression model. In this model, the observed change from baseline in PCL-5 was the dependent variable, and the predictors were PCL-5 scores measured at other time points and randomization group. By generating 20 imputed datasets for each missing value, the variability between these imputed values reflects the uncertainty about the missing data. The 20 complete datasets were analyzed as described below and the results from the 20 complete datasets were pooled to produce an estimate for the primary endpoint (for ITT and ITTa populations).

An independent samples t-tests was used to compare the change from baseline in PCL-5 scores at 12 weeks between the randomization groups. The dependent variable was the change in PCL-5 score from baseline, and the independent variable was randomization group (e.g., VeNS vs. sham).

To assess the longitudinal effects, a repeated measures ANOVA (RM ANOVA) was performed to examine how PCL-5 score changes during the study period and to determine if these changes differ between the two randomization groups. The within-subject was time (four timepoints at which PCL-5 scores were collected: week 0 (baseline), week 4, week 8 and week 12) and the between-subject factor was randomization group (VeNS and Sham). The assumption of sphericity was assessed using the Mauchly's test of sphericity. A significant group-by-time interaction was obtained. Post hoc comparisons were undertaken using paired sample t-tests for within group comparisons (change in PCL-5 score from baseline to each time point for each randomization group) and independent samples t-test for between group comparisons. A Bonferroni correction was applied to adjust the alpha level for multiple comparisons.

Results: A total of 383 participants aged 22 to 80 years old who had been previously diagnosed with PTSD by a medical practitioner (PCL-5 score of 31 or above) were randomized to the VeNS device group (n=191) or the Sham group (n=192). 201 participants completed the study (VeNS, n=108; sham, n=93). Of those who were counted as withdrawals (n=182), 62 withdrew prior to the baseline visit.

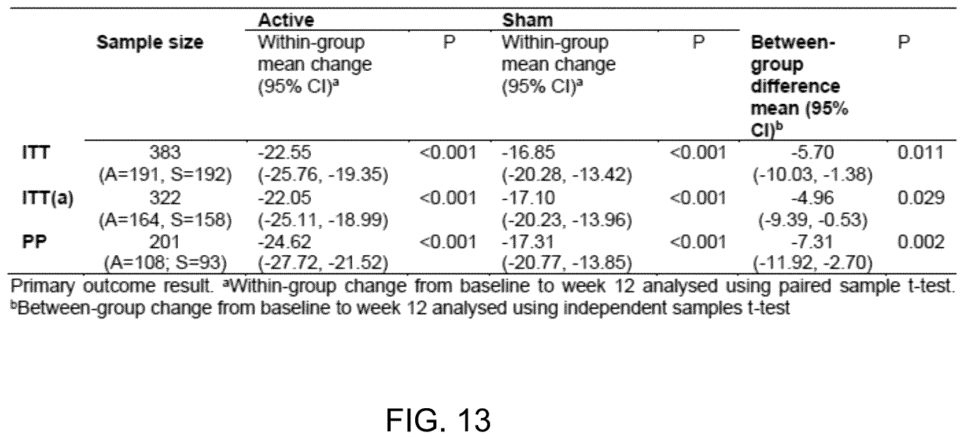

The table in FIG. 13 provides the effect of VeNS (Active) on PCL-5 scores from baseline to 12 weeks compared with the sham (ITT and PP analyses). All statistical approaches (ITT, ITTa, and PP) demonstrate that the VeNS (Active) group achieved a significantly greater reduction in PCL-5 score from baseline to week 12 compared with the sham group. In the Active and Sham columns, within-group change from baseline to week 12 was analyzed using paired sample t-test. In the right-hand column, between-group change from baseline to week 12 was analyzed using independent samples t-test.

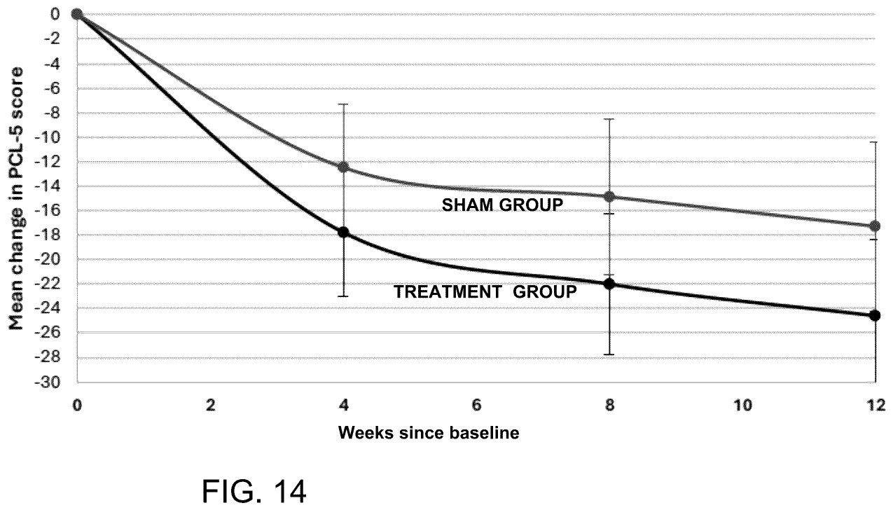

The mean change in PCL-5 score is presented graphically over time (baseline, weeks 4, 8 and 12) and by randomization group (PP population) in FIG. 14. As the graph indicates, there is clear divergence between the two groups at the first data point at week 4 from baseline and continued expanding through week 12 of the study.

Results from a RM ANOVA (PP analysis) showed a group-by-time interaction effect on change in PCL-5 score from baseline to week 12 (F (2.14, 199)=7.02, p<0.001, η²=0.034). Post hoc analyses indicate that at each time point, the VeNS (Active) group reported significantly greater reductions in their PCL-5 score from baseline, compared with the sham group (Bonferroni adjustment).

While the foregoing study was terminated after week 12, an extended treatment period could potentially be continued indefinitely (months or years) as a maintenance regimen (with periodic supervision and evaluation), or until the subject or the supervising medical professional determines that no further improvement is being achieved. Accordingly, the definition of “extended treatment period” is not limited to 12 weeks but would be continued for multiple weeks until a desired level of improvement in the subject's symptoms has been achieved.

The above description of the disclosed embodiments is provided to enable a person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A method of treating an anxiety-related disorder in a human subject through delivery of vestibular nerve stimulation (VeNS), the method comprising:
positioning at least one electrode into electrical contact with the human subject and proximate to a location of the subject's vestibular system; and
delivering VeNS to the human subject from a current source connected with the at least one electrode, the current source configured to deliver an alternating polarity square wave current at a frequency of approximately 0.25 Hz with an approximately 50% duty cycle for a predetermined treatment session.

2. The method of claim 1, wherein positioning and delivering are performed before, during or after an anxiety-inducing event.

3. The method of claim 1, wherein positioning and delivering are performed daily for an extended treatment period, wherein the extended treatment period extends until a desired level of improvement in one or more disorder symptom experienced by the human subject has been achieved.

4. The method of claim 1, wherein the predetermined treatment session is from 30 minutes to 60 minutes.

5. The method of claim 1, where the anxiety-related disorder is post-traumatic stress disorder (PTSD).

6. The method of claim 1, wherein the square wave current has a range of 0.01 mA to 1.5 mA.

7. The method of claim 1, wherein the at least one electrode comprises two electrodes, wherein the two electrodes are configured to be releasably affixed behind the human subject's left and right ears.

8. A method of treating a stress-related disorder using vestibular nerve stimulation (VeNS), comprising:
daily, for a treatment session, positioning an electrode into electrical contact with a subject's scalp adjacent to each of the subject's left and right mastoid process; and
delivering VeNS to the subject from a current source connected with the electrodes, the current source configured to deliver an alternating polarity current within a range of 0.01 mA to 1.5 mA and a frequency of 0.25 Hz with an approximately 50% duty cycle; and
after the treatment session, removing the electrodes from the human subject.

9. The method of claim 8, wherein the treatment session is from 30 minutes to 60 minutes.

10. The method of claim 8, wherein the treatment session is 30 minutes.

11. The method of claim 8, wherein the alternating polarity current is a square wave current having a range of 1 mA to 1.5 mA.

12. The method of claim 8, wherein positioning and delivering are performed daily for an extended treatment period until a desired level of improvement in one or more disorder symptom experienced by the subject has been achieved.

13. The method of claim 8, where the stress-related disorder is post-traumatic stress disorder (PTSD).

14. The method of claim 8, wherein the electrodes each comprise an anode and a cathode disposed on a single stimulation pad, wherein delivering VeNS comprises using a circular flow pattern from the anode to the cathode across the mastoid process.

15. The method of claim 8, wherein the electrodes comprise first and second binaural stimulation pads, wherein positioning comprises disposing the first binaural stimulation pad adjacent a left mastoid process and the second binaural stimulation pad adjacent a right mastoid process, and wherein the current source is synchronized to simultaneously deliver VeNS to each binaural stimulation pad.
